(19) United States
(12) Patent Application Publication
(10) Pub. No.: US 2024/0100126 A1
(43) Pub. Date: Mar. 28, 2024

(54) Methods Comprising Continuous Administration of a GLP-1 Receptor Agonist and Co-administration of a Drug

(71) Applicant: i2O Therapeutics, Inc., Cambridge, MA (US)

(72) Inventors: Andrew YOUNG, Cambridge, MA (US); Michelle BARON, Cambridge, MA (US)

(21) Appl. No.: 18/319,010

(22) Filed: May 17, 2023

Related U.S. Application Data
(63) Continuation of application No. 17/069,521, filed on Oct. 13, 2020, now Pat. No. 11,654,183.
Application No. 17/069,521 is a continuation of application No. 15/861,258, filed on Jan. 3, 2018, now Pat. No. 10,835,580.
(60) Provisional application No. 62/441,833, filed on Jan. 3, 2017.

(51) Int. Cl. A61K 38/26; A61K 9/00; A61K 31/167; A61K 31/352; A61K 31/37; A61K 31/40; A61K 31/401; A61K 31/565; A61K 31/567; A61K 31/7048; A61K 38/05; A61P 3/04; A61P 3/10; A61P 7/02; A61P 9/00; A61P 15/18; A61P 25/00; A61P 25/04

(52) U.S. Cl. CPC A61K 38/26; A61K 9/0019; A61K 9/0053; A61K 31/167; A61K 31/352; A61K 31/37; A61K 31/40; A61K 31/401; A61K 31/565; A61K 31/567; A61K 31/7048; A61K 38/05; A61P 3/04; A61P 3/10; A61P 7/02; A61P 9/00; A61P 15/18; A61P 25/00; A61P 25/04

(57) ABSTRACT

Provided is a method for administering to a subject, via an implantable delivery device, a continuous subcutaneous dose of glucagon-like peptide-1 (GLP-1) analog, where the subject is orally co-administered a drug after implantation of the implantable delivery device and during continuous subcutaneous dosing of the GLP-1 analog.

### CROSS REFERENCE TO RELATED APPLICATIONS

This application is a U.S. continuation of U.S. application Ser. No. 17/069,521, filed on Oct. 13, 2020, which application is a U.S. continuation application of U.S. application Ser. No. 15/861,258, filed Jan. 3, 2018, now U.S. Pat. No. 10,835,580, which application claims priority to and the benefit of U.S. Provisional Application No. 62/441,833, filed Jan. 3, 2017, which applications are herein incorporated by reference in their entirety.

### SEQUENCE LISTING

The instant application contains a Sequence Listing which has been submitted in XML format and is hereby incorporated by reference in its entirety. Said XML sequence listing, created on May 16, 2023 is named 743085_IOT-052CON2_SL.xml and is about 1.99 kilobytes in size.

### BACKGROUND

By some estimates, over 350 million people worldwide are presently diagnosed with type 2 diabetes mellitus (T2D) and one in three people in the United States will develop T2D in their lifetime. For treatment of this disease, the American Diabetes Association (ADA) recommends metformin as first-line therapy due to its low cost, availability and reasonable efficacy in reducing glycated hemoglobin (HbA1c), despite certain shortcomings associated with this drug. The ADA also recommends potential second-line options, including glucagon-like peptide-1 (GLP-1) receptor agonists, sodium-glucose cotransporter 2 (SGLT2) inhibitors, dipeptidyl peptidase-4 inhibitors (DPP-4), sulfonylureas, thiazolidinediones and insulin. Treatment of T2D with GLP-1 receptor agonist peptides, in particular, has grown. GLP-1 receptor agonists generally provide important effects in subjects beyond blood glucose control, such as effecting weight loss, preserving beta-cell function, and mitigating hypertension, hypoglycemia and/or hyperlipidemia. Methods are presently needed to more fully and properly implement treatment with GLP-1 receptor agonists and better address growing needs of subjects with T2D, obesity or excessive body weight, some of whom must simultaneously manage treatment of unrelated diseases or disorders.

### SUMMARY

Periodic and subcutaneous administrations (i.e., injections) of a GLP-1 receptor agonist are presently used to achieve a glucose-dependent increase in insulin in subjects with T2D. The present invention encompasses the recognition of a problem regarding treatment of T2D with GLP-1 receptor agonists. Specifically, injections of certain GLP-1 receptor agonists generally slow gastric emptying and can reduce the extent and rate of absorption of orally administered drugs. Upon injection of certain GLP-1 receptor agonists, co-administration of certain drugs for treatment of diseases other than T2D may require dose adjustment of these drugs (relative to doses prescribed for the drugs when administered alone) or preclude co-administration of certain drugs upon injection of the GLP-1 receptor agonists. Certain injectable GLP-1 receptor agonists have been found to distort areas under the curve (AUC), Cₘₐₓ, and Tₘₐₓ for certain orally available drugs for treatment of diseases, disorders or conditions unrelated to T2D upon co-administration. Consequently, since doses adjustments are often impractical, such drugs must be administered before (e.g., at least one hour prior to) injection of the GLP-1 receptor agonist.

For example, according to prescribing information (PI) for injectable Byetta® (exenatide) for the treatment of T2D, “[oral contraceptive] OC products should be administered at least one hour prior to BYETTA injection.” As explained in the PI for Byetta®, co-administration of an oral contraceptive and Byetta® results in decreased Cₘₐₓ and delayed Tₘₐₓ for the oral contraceptive: “The effect of BYETTA (10 mcg BID) on single and on multiple doses of a combination oral contraceptive (35 mcg ethinyl estradiol plus 150 mcg levonorgestrel) was studied in healthy female subjects. Repeated daily doses of the oral contraceptive (OC) given 30 minutes after BYETTA administration decreased the Cₘₐₓ of ethinyl estradiol and levonorgestrel by 45% and 27%, respectively and delayed the Tₘₐₓ of ethinyl estradiol and levonorgestrel by 3.0 hours and 3.5 hours, respectively, as compared to the oral contraceptive administered alone. Administration of repeated daily doses of the OC one hour prior to BYETTA administration decreased the mean Cₘₐₓ of ethinyl estradiol by 15% but the mean Cₘₐₓ of levonorgestrel was not significantly changed as compared to when the OC was given alone.”

Also according to prescribing information (PI) for injectable Byetta® (exenatide) for the treatment of T2D, “[a]cetaminophen AUC, Cₘₐₓ and Tₘₐₓ were not significantly changed when acetaminophen was given 1 hour before BYETTA injection.” However, a s explained in the PI for Byetta®, co-administration of a pain reliever such as acetaminophen with Byetta®, or after Byetta® injection, results in decreased areas under the curve (AUC) and Cₘₐₓ, and increases in Tₘₐₓ, for acetaminophen. “When 1000 mg acetaminophen elixir was given with 10 mcg BYETTA (0 h) and 1 hour, 2 hours, and 4 hours after BYETTA injection, acetaminophen AUCs were decreased by 21%, 23%, 24%, and 14%, respectively; Cₘₐₓ was decreased by 37%, 56%, 54%, and 41%, respectively; Tₘₐₓ was increased [delayed] from 0.6 hour in the control period to 0.9 hour, 4.2 hours, 3.3 hours, and 1.6 hours, respectively.”

Unfortunately, real life circumstances often preclude subjects (i.e., human subjects) from adhering to prescribing information regarding pre-administration of drugs for treatment(s) unrelated to T2D prior to injection of a GLP-1 receptor agonist for the treatment of T2D. GLP-1 receptor agonists include twice-daily injectable Byetta® (exenatide), once-daily injectable Victoza® (liraglutide), once weekly injectable Trulicity® (dulaglutide) and once weekly injectable Ozempic® (semaglutide). Specifically, real life onset of conditions such as pain, heart attack, hypertension, stroke, blood clot, or the need for contraception commonly occur after, sometimes immediately after, bolus injection of a GLP-1 receptor agonist. Yet, when confronted with such circumstances, the subject must delay treatment until one or several hours before administration of the next injection of GLP-1 receptor agonist. Failure to adhere to this prescribing information, as it relates to pre-administration of such drugs before bolus injection of the GLP-1 receptor agonist, puts subjects at risk of effecting suboptimal AUC, Cₘₐₓ and/or Tₘₐₓ of such drugs.

It has been discovered that continuous administration of GLP-1 receptor agonists, such as exenatide, via an implantable delivery device is not accompanied by either substantial delays in gastric emptying (See FIGS. 1 & 2) or substantial reductions in blood concentrations of glucagon (See FIGS. 3-5). Without being bound by theory, it thus appears that delays in gastric emptying and reductions in blood concentrations of glucagon are substantially attributable to the mode of administration for certain GLP-1 receptor agonists.

It has also been discovered that certain drugs other than those for treating T2D (e.g., drugs for treatment or prevention of pain, conditions associated with heart disease or a heart attack, hypertension, stroke or blood clot, and oral contraceptives) can effectively be co-administered upon continuous administration of a GLP-1 receptor agonist via an implantable delivery device. Therefore, the requirement for pre-administration of certain drugs, relative to injection of the GLP-1 receptor agonist such as exenatide, similarly appear attributable to the mode of administration for the GLP-1 receptor agonist.

Thus, whereas bolus injection of a GLP-1 receptor agonist such as Byetta® require advance oral administration of certain drugs (e.g., for treatment or prevention of pain and oral contraceptives) at least one hour prior to injection of Byetta®, applicants have discovered that such drugs can be orally administered after implantation of an osmotic delivery device and during continuous subcutaneous delivery (e.g., during three, six, twelve, or twenty-four month administration periods) of a GLP-1 analog such as exenatide (e.g., at 20 μg/day or 60 μg/day ITCA-650). This increased versatility of co-administration provides subjects, who have been administered implantable osmotic delivery devices for continuous subcutaneous delivery of a GLP-1 analog, with the option to effectively co-administer orally available drugs (e.g., for treatment of pain, a heart condition, heart attack, hypertension, stroke, and/or preventing a blood clot or providing contraception) at any time during three, six, twelve, or twenty-four month administration period of continuous subcutaneous delivery of the GLP-1 analog.

In certain embodiments, the present invention provides a method for administering to a subject, via an implantable delivery device, a continuous subcutaneous dose of glucagon-like peptide-1 (GLP-1) analog, where the subject is orally co-administered a drug after implantation of the implantable delivery device and during continuous subcutaneous dosing of the GLP-1 analog. In other words, the subject is co-administered the drug following implantation of the implantable delivery device and during three, six, twelve, or twenty-four month administration period of continuous subcutaneous delivery of the GLP-1 analog without resorting to advance administration of the drug prior to administration (i.e., implantation) of the GLP-1 analog.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the invention will be apparent from the following detailed description and claims.

### BRIEF DESCRIPTIONS OF THE DRAWINGS

The above and further features will be more clearly appreciated in view of the following detailed description and accompanying drawings.

FIG. 1 is a graph illustrating 0-30-minute increments in plasma glucose levels during test meals for 10-, 20-, 40- and 80 μg/day exenatide treatments, measured before and after 5, 15, and 29 days of treatment. Symbols are group means of individual increments±standard error of the mean (SEM).

FIG. 2 is a graph illustrating dose-responses for 30-minute changes in glucose concentrations during test meals relative to pre-treatment values. Curves for Days 5, 15 and 29 are 3-parameter sigmoids constrained to share a common effective dose causing 50% inhibition (ED₅₀). Symbols are group means of individual values±SEM.

FIG. 3 depicts graphs illustrating plasma glucagon profiles during meal tolerance tests plotted according to duration of treatment (different symbols and colors) for each of the 4 dose groups (separate panels). Symbols are means±SEM for data present at each condition.

FIG. 4 depicts graphs illustrating changes in plasma glucagon concentration from pre-meal values during a test meal. Symbols, colors and layout have the same meanings as those in FIG. 3.

FIG. 5 depicts graphs illustrating integrated glucagon concentrations (left panel) or glucagon changes (right panel) during Meal Tolerance Test (MTT) as a function of duration of treatment for each dose group.

FIG. 6A (left), redrawn from Saad et al., is a graph illustrating changing [insulin] vs [glucose] relationship during the progression from normal glucose tolerance to T2D.

FIG. 6B (right) is a graph that exemplifies the diverse [insulin] vs [glucose] relationships in the current study.

FIG. 7 is a graph illustrating multiples above pre-treatment baseline of best fitting [insulin]×[glucose] slopes. The curves are the best fitting exponential association as a function of duration of treatment.

FIG. 8 is a graph illustrating dose response for the effect of ITCA-650 to increase slope of the [insulin]/[glucose] relationship.

FIG. 9 is a graph illustrating mean plasma concentrations of acetaminophen over time, at day 27, alone and upon co-administration with ITCA-650, during continuous delivery of exenatide via an implanted osmotic delivery device.

FIG. 10 provides statistical assessments of drug-drug interactions of exenatide and ethinyl estradiol (EE) and levonorgestrel (LNG) from Levora® (OC) during continuous delivery of exenatide via an implanted osmotic delivery device.

FIG. 11 is a chart that illustrates pharmacokinetic parameters demonstrating that ITCA-650 did not substantially affect pharmacokinetics of certain orally co-administered medications to a clinically relevant degree.

### DETAILED DESCRIPTION

### Definitions

Glucagon-like peptide-1 (GLP-1) derives from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. GLP-1 is produced as a 37-amino acid peptide that corresponds to amino acids 72 through 108 of proglucagon (92 to 128 of preproglucagon). GLP-1(7-36) amide or GLP-1(7-37) acid are biologically active forms of GLP-1, that demonstrate essentially equivalent activity at the GLP-1 receptor.

GLP-1 and GLP-1 analogs, acting as agonists at the GLP-1 receptor, have been shown to provide effective hypoglycemic control, e.g., for treating patients with type-2 diabetes. Certain GLP-1 analogs are being sold or are in development for treatment of type-2 diabetes including, e.g., Byetta® & Bydureon BCise® (exenatide), Ozempic® (semaglutide), Victoza® (liraglutide), Adlyxin® (lixisenatide); Tanzeum® (albiglutide), and Trulicity® (dulaglutide).

The term “osmotic delivery device” as used herein typically refers to a device used for delivery of a drug (e.g., an insulinotrophic peptide) to a subject, wherein the device comprises, for example, a reservoir (made, e.g., from a titanium alloy) having a lumen that contains a suspension formulation comprising a drug (e.g., an insulinotrophic peptide) and an osmotic agent formulation. A piston assembly positioned in the lumen isolates the suspension formulation from the osmotic agent formulation. A semi-permeable membrane is positioned at a first distal end of the reservoir adjacent the osmotic agent formulation and a diffusion moderator (which defines a delivery orifice through which the suspension formulation exits the device) is positioned at a second distal end of the reservoir adjacent the suspension formulation. Typically, the osmotic delivery device is implanted within the subject, for example, subdermally or subcutaneously (e.g., in the abdominal area or in the inside, outside, or back of the upper arm). An exemplary osmotic delivery device is the DUROS® delivery device. Examples of terms synonymous to “osmotic delivery device” include but are not limited to “osmotic drug delivery device,” “osmotic drug delivery system,” “osmotic device,” “osmotic delivery device,” “osmotic delivery system,” “osmotic pump,” “implantable drug delivery device,” “drug delivery system,” “drug delivery device,” “implantable osmotic pump,” “implantable drug delivery system,” and “implantable delivery system.” Other terms for “osmotic delivery device” are known in the art. As used herein, “ITCA 650” is an osmotic delivery device comprising exenatide having the amino acid sequence of SEQ ID NO: 1: H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly- Ala-Pro-Pro-Pro-Ser-NH₂.

The term “continuous delivery” as used herein typically refers to a substantially continuous release of drug from an osmotic delivery device and into tissues near the implantation site, e.g., subdermal and subcutaneous tissues. For example, the osmotic delivery device releases drug essentially at a predetermined rate based on the principle of osmosis. Extracellular fluid enters the osmotic device through the semi-permeable membrane directly into the osmotic engine that expands to drive the piston at a slow and consistent rate of travel. Movement of the piston forces the drug formulation to be released through the orifice of the diffusion moderator. Thus, release of the drug from the osmotic delivery device is at a slow, controlled, consistent rate.

The term “substantial steady-state delivery” as used herein typically refers to delivery of a drug at or near a target concentration over a defined period of time, wherein the amount of the drug being delivered from an osmotic delivery device is substantially zero-order delivery. Substantial zero-order delivery of a therapeutic agent (e.g., an insulinotrophic peptide, preferably, an exenatide) means that the rate of drug delivered is constant and is independent of the drug available in the delivery system; for example, for zero-order delivery, if the rate of drug delivered is graphed against time and a line is fitted to the data the line has a slope of approximately zero, as determined by standard methods (e.g., linear regression).

As used herein, the terms “treatment,” “treat,” and “treating” refer to reversing, alleviating, ameliorating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “subject,” as used herein, means an animal, preferably a mammal, and most preferably a human. The term “subject,” as used herein, also means a patient, preferably a human patient suffering from T2D, obesity or in need of weight loss.

As used herein, the term “co-administration” generally refers to separate administration of a drug to a subject during or after bolus injection of GLP-1 receptor agonist to the subject, or separate administration of a drug to a subject during or after insertion in the subject of an osmotic delivery device comprising GLP-1 receptor agonist such as exenatide.

The term “dose adjustment” refers to a change in dosage of a drug for treatment of a disease or disorder other than type-2 diabetes that is made upon co-administration of a GLP-1 receptor agonist, relative to the dosage used upon administration of the drug alone or in the absence of the GLP-1 receptor agonist.

Unless otherwise defined, all technical and scientific terms used herein have the same meaning as commonly understood by one of ordinary skill in the art to which this invention belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

### Description of Exemplary Embodiments

In one aspect, the present invention provides a method comprising administering to a subject, via an implantable delivery device, a continuous subcutaneous dose of glucagon-like peptide-1 (GLP-1) analog, where the subject is orally co-administered a drug after implantation of the implantable delivery device and during continuous subcutaneous dosing of the GLP-1 analog.

In another aspect, the present invention provides a drug for use in a method of treatment of a subject (e.g., a patient suffering from T2D and/or obesity and/or in need of weight loss), the method comprising administering to the subject (e.g., patient), via an implantable osmotic delivery device, a continuous subcutaneous dose of a glucagon-like peptide-1 (GLP-1) analog; and orally co-administering a drug after implantation of the implantable delivery device and during continuous subcutaneous dosing of the GLP-1 analog.

In some embodiments, the subject is orally co-administered a drug one hour to six months after implantation of the implantable delivery device. In some embodiments, the subject is orally co-administered a drug one hour to twenty-four hours after implantation of the implantable delivery device. In some embodiments, the subject is orally co-administered a drug one day to seven days after implantation of the implantable delivery device. In some embodiments, the subject is orally co-administered a drug one week to one month after implantation of the implantable delivery device. In some embodiments, the subject is orally co-administered a drug one month to three months after implantation of the implantable delivery device. In some embodiments, the subject is orally co-administered a drug three months to six months after implantation of the implantable delivery device. In some embodiments, the subject is orally co-administered a drug six months to one year after implantation of the implantable delivery device. In some embodiments, the subject is orally co-administered a drug one year to two years after implantation of the implantable delivery device.

In some embodiments, the drug is administered for treatment of a disease or disorder other than type-2 diabetes. In some embodiments, the disease or disorder other than type-2 diabetes is selected from the group consisting of pain, elevated blood levels of cholesterol, heart disease, hypertension, heart attack, stroke or blood clot.

In some embodiments, the drug is a contraceptive administered to prevent conception of a child.

In some embodiments, the drug is selected from the group consisting of acetaminophen, atorvastatin, lisinopril, digoxin, ethinyl estradiol, levonorgestrel, R-warfarin, and/or S-warfarin.

In some embodiments, the drug is a pain reliever, such as acetaminophen.

In some embodiments, the drug is acetaminophen and the ratio of the AUC for co-administered acetaminophen after implantation of the implantable delivery device and during continuous subcutaneous dosing of the GLP-1 analog relative to reference AUC for acetaminophen administered alone is between 1.0 and 1.25 or between 0.75 and 1.25.

In some embodiments, the drug is acetaminophen and the AUC for co-administered acetaminophen (e.g., co-administered within 1, 2 or 4 hours of implantation) and during continuous subcutaneous dosing of the GLP-1 analog are reduced less than 10% or 5% relative to reference AUC for acetaminophen administered alone.

In some embodiments, the drug is acetaminophen and the ratio of the Cₘₐₓ for co-administered acetaminophen after implantation of the implantable delivery device and during continuous subcutaneous dosing of the GLP-1 analog relative to reference Cₘₐₓ for acetaminophen administered alone is between 1.0 and 1.25 or between 0.75 and 1.25.

In some embodiments, the drug is acetaminophen and the Cₘₐₓ for co-administered acetaminophen (e.g., within 1, 2 or 4 hours of implantation) and during continuous subcutaneous dosing of the GLP-1 analog are reduced less than 30%, 20%, 10% or 5% relative to reference Cₘₐₓ for acetaminophen administered alone.

In some embodiments, the drug is acetaminophen and the Tₘₐₓ for co-administered acetaminophen (e.g., within 1, 2 or 4 hours of implantation) and during continuous subcutaneous dosing of the GLP-1 analog is increased by less than 2 hours or 1 hour relative to reference Tₘₐₓ for acetaminophen administered alone.

In some embodiments, the drug is an oral contraceptive, such as ethinyl estradiol and/or levonorgestrel. In some embodiments, the oral contraceptive is a combination of ethinyl estradiol and levonorgestrel (e.g., Levora®, 35 mcg ethinyl estradiol plus 150 mcg levonorgestrel).

In some embodiments, the drug is ethinyl estradiol and/or levonorgestrel and the ratio of the AUC for co-administered ethinyl estradiol and/or levonorgestrel after implantation of the implantable delivery device and during continuous subcutaneous dosing of the GLP-1 analog relative to reference AUC for ethinyl estradiol and/or levonorgestrel administered alone is between 0.75 and 1.25 or between 0.75 and 1.50.

In some embodiments, the drug is ethinyl estradiol and/or levonorgestrel and the ratio of the Cₘₐₓ for co-administered ethinyl estradiol and/or levonorgestrel after implantation of the implantable delivery device and during continuous subcutaneous dosing of the GLP-1 analog relative to reference Cₘₐₓ for ethinyl estradiol and/or levonorgestrel administered alone is between 0.75 and 1.25 or between 0.75 and 1.50.

In some embodiments, the drug is ethinyl estradiol and/or levonorgestrel and the Cₘₐₓ for co-administered ethinyl estradiol and/or levonorgestrel (e.g., within 1, 2 or 4 hours of implantation) and during continuous subcutaneous dosing of the GLP-1 analog are reduced less than 30%, 20%, 10% or 5% relative to reference Cₘₐₓ for ethinyl estradiol and/or levonorgestrel administered alone.

In some embodiments, the drug is ethinyl estradiol and/or levonorgestrel and the Tₘₐₓ for co-administered ethinyl estradiol and/or levonorgestrel (e.g., within 1, 2 or 4 hours of implantation) and during continuous subcutaneous dosing of the GLP-1 analog is increased less than 3 hours, 2 hours or 1 hour relative to reference Tₘₐₓ for ethinyl estradiol and/or levonorgestrel administered alone.

In some embodiments, the drug is for the treatment or prevention of elevated blood levels of cholesterol. In some embodiments, the drug is a statin. In some embodiments, the drug is atorvastatin.

In some embodiments, the drug is atorvastatin and the ratio of the AUC for co-administered atorvastatin after implantation of the implantable delivery device and during continuous subcutaneous dosing of the GLP-1 analog relative to reference AUC for atorvastatin administered alone is between 1.0 and 1.25 or between 1.0 and 1.50.

In some embodiments, the drug is atorvastatin and the ratio of the Cₘₐₓ for co-administered atorvastatin after implantation of the implantable delivery device and during continuous subcutaneous dosing of the GLP-1 analog relative to reference Cₘₐₓ for atorvastatin administered alone is between 1.0 and 1.5 or between 1.0 and 1.75.

In some embodiments, the drug is for the treatment or prevention of hypertension and/or heart disease. In some embodiments, the drug is digoxin.

In some embodiments, the drug is digoxin and the ratio of the AUC for co-administered digoxin after implantation of the implantable delivery device and during continuous subcutaneous dosing of the GLP-1 analog relative to reference AUC for digoxin administered alone is between 1.0 and 1.25 or between 1.0 and 1.50.

In some embodiments, the drug is digoxin and the ratio of the Cₘₐₓ for co-administered digoxin after implantation of the implantable delivery device and during continuous subcutaneous dosing of the GLP-1 analog relative to reference Cₘₐₓ for digoxin administered alone is between 1.0 and 1.25 or between 1.0 and 1.50.

In some embodiments, the drug is an angiotensin converting enzyme (ACE) inhibitor. In some embodiments, the drug is lisinopril.

In some embodiments, the drug is lisinopril and the ratio of the AUC for co-administered lisinopril after implantation of the implantable delivery device and during continuous subcutaneous dosing of the GLP-1 analog relative to reference AUC for lisinopril administered alone is between 1.5 and 2.0 or between 1.0 and 2.0.

In some embodiments, the drug is lisinopril and the ratio of the Cₘₐₓ for co-administered lisinopril after implantation of the implantable delivery device and during continuous subcutaneous dosing of the GLP-1 analog relative to reference Cₘₐₓ for lisinopril administered alone is between 1.25 and 1.75 or between 1.0 and 2.0.

In some embodiments, the drug is for the treatment or prevention of a heart attack, stroke, and/or blood clot. In some embodiments, the drug is an anticoagulant. In some embodiments, the drug is R-warfarin and/or S-warfarin.

In some embodiments, the drug is R-warfarin and/or S-warfarin and the ratio of the AUC for co-administered R-warfarin and/or S-warfarin after implantation of the implantable delivery device and during continuous subcutaneous dosing of the GLP-1 analog relative to reference AUC for R-warfarin and/or S-warfarin administered alone is between 1.0 and 1.25 or between 0.75 and 1.5.

In some embodiments, the drug is R-warfarin and/or S-warfarin and the ratio of the Cₘₐₓ for co-administered R-warfarin and/or S-warfarin after implantation of the implantable delivery device and during continuous subcutaneous dosing of the GLP-1 analog relative to reference Cₘₐₓ for R-warfarin and/or S-warfarin administered alone is less than 1.5 or 1.25.

In some embodiments, the drug is co-administered without dose adjustment. In other words, the normally prescribed dose for the drug is not changed after implantation of the delivery device and during continuous subcutaneous dosing of the GLP-1 analog.

In some embodiments, the drug is self-administered by the subject. In other words, the drug, either prescribed by a physician or obtained as an over-the-counter drug, is taken orally by the subject.

In another aspect, the present invention provides a method comprising administering to a subject, via an implantable delivery device, a continuous subcutaneous dose of glucagon-like peptide-1 (GLP-1) analog, without providing a substantial delay in a rate of gastric emptying in the subject, following administration, relative to the rate of gastric emptying for the subject prior to administration.

In another aspect, the present invention provides a drug for use in a method of treatment of a subject (e.g., a patient suffering from T2D and/or obesity and/or in need of weight loss), the method comprising administering to the subject (e.g., patient), via an implantable osmotic delivery device, a continuous subcutaneous dose of a glucagon-like peptide-1 (GLP-1) analog without providing a substantial delay in a rate of gastric emptying in the subject, following administration, relative to the rate of gastric emptying for the subject prior to administration.

In some embodiments, the method provides less than 20% delay in the rate of gastric emptying in the subject, following administration, relative to the rate of gastric emptying for the subject prior to administration. In some embodiments, the method provides less than 10%, 5% or 1% delay in the rate of gastric emptying in the subject, following administration, relative to the rate of gastric emptying for the subject prior to administration.

In some embodiments, the method provides no substantial delay in the rate of gastric emptying in the subject, between 5 and 29 days following administration, relative to the rate of gastric emptying for the subject prior to administration. In some embodiments, the method provides no substantial delay in a rate of gastric emptying in the subject, between 1 day and 1 week, between 1 day and 2 weeks, or between 1 day and 1 month, following administration, relative to the rate of gastric emptying for the subject prior to administration. In some embodiments, the method provides no substantial delay in a rate of gastric emptying in the subject, during continuous subcutaneous delivery (e.g., during three, six, twelve, or twenty-four month administration period) of a GLP-1 analog such as exenatide (e.g. ITCA-650 at 20 μg/day exenatide or ITCA-650 60 μg/day exenatide).

In some embodiments, the method provides no substantial delay in the fasting rate of gastric emptying. Fasting conditions (e.g., those within a fasting period of at least 24, 12, 8, 6, 4 or 2 hours without consumption of food or a meal) correspond to those well known to those of ordinary skill in the art. As used herein, the term “substantial” corresponds to less than 20%, less than 10%, less than 5% or less than 1%.

In some embodiments, the method provides no substantial (e.g., less than 20%, less than 10%, less than 5% or less than 1%) delay in the post-prandial rate of gastric emptying. Post-prandial conditions (e.g., those within a feeding period of 12, 8, 6, 4, 2 or 1 hour(s), during which food or a meal was consumed) correspond to those well known to those of ordinary skill in the art.

In another aspect, the present invention provides a method comprising administering to a subject, via an implantable delivery device, a continuous subcutaneous dose of glucagon-like peptide-1 (GLP-1) analog without effecting a substantial reduction in glucagon concentration in blood of the subject, following administration, relative to glucagon concentration in blood of the subject prior to administration.

In another aspect, the present invention provides a drug for use in a method of treatment of a subject (e.g., a patient suffering from T2D and/or obesity and/or in need of weight loss), the method comprising administering to the subject (e.g., patient), via an implantable osmotic delivery device, a continuous subcutaneous dose of a glucagon-like peptide-1 (GLP-1) analog without providing a substantial reduction in glucagon concentration in blood of the subject, following administration, relative to glucagon concentration in blood of the subject prior to administration.

In some embodiments, the method provides less than 20% reduction in glucagon concentration in blood of the subject, following administration, relative to glucagon concentration in blood of the subject prior to administration. In some embodiments, the method provides less than 10%, 5% or 1% reduction in glucagon concentration in blood of the subject, following administration, relative to glucagon concentration in blood of the subject prior to administration.

In some embodiments, the method provides no substantial reduction in glucagon concentration in blood of the subject, between 5 and 29 days following administration, relative to glucagon concentration in blood of the subject prior to administration. In some embodiments, the method provides no substantial reduction in glucagon concentration in blood of the subject, between 1 day and 1 week, between 1 day and 2 weeks, or between 1 day and 1 month, following administration, relative to glucagon concentration in blood of the subject prior to administration. In some embodiments, the method provides no substantial reduction in glucagon concentration in blood of the subject, during continuous subcutaneous delivery (e.g., during three, six, twelve, or twenty-four month administration period) of a GLP-1 analog such as exenatide (e.g. ITCA-650 at 20 μg/day exenatide or ITCA-650 60 μg/day exenatide).

In some embodiments, the method provides no substantial (e.g., less than 20%, less than 10%, less than 5% or less than 1%) reduction in fasting glucagon concentration.

In some embodiments, the method provides no substantial (e.g., less than 20%, less than 10%, less than 5% or less than 1%) reduction in post-prandial glucagon concentration.

In some embodiments, the GLP-1 analog is exenatide. In some embodiments, the GLP-1 analog is other than exenatide. In some embodiments, the GLP-1 analog is selected from the group consisting of Ozempic® (semaglutide), Victoza® (liraglutide), Adlyxin® (lixisenatide), Tanzeum® (albiglutide), and Trulicity® (dulaglutide). In some embodiments, the GLP-1 analog is Ozempic® (semaglutide). In some embodiments, the GLP-1 analog is Victoza® (liraglutide). In some embodiments, the GLP-1 analog is Adlyxin® (lixisenatide). In some embodiments, the GLP-1 analog is Trulicity® (dulaglutide). In some embodiments, the GLP-1 analog is Tanzeum® (albiglutide).

In some embodiments, the GLP-1 analog is administered for treatment of a metabolic disorder. In some embodiments, the GLP-1 analog is administered for treatment of a type 2 diabetes mellitus. In some embodiments, the GLP-1 analog is administered for treatment of obesity. In some embodiments, the GLP-1 analog is administered for effecting weight loss in the subject.

In some embodiments, the subject is administered a dose of 20 μg/day ITCA-650. In some embodiments, the subject is administered a dose of 60 μg/day ITCA-650.

In some embodiments, the subject is human.

### EXEMPLIFICATION

The following examples are put forth to provide those of ordinary skill in the art with a complete disclosure and description of how to practice the present invention, and are not intended to limit the scope of what the inventors regard as the invention. Efforts have been made to ensure accuracy with respect to numbers used (e.g., amounts, concentrations, and percent changes) but some experimental errors and deviations may remain.

### General Methods for Examples 1-3

Data source: Data relating to the Meal Tolerance Test (MTT) were derived from the evaluable cohort, comprising all randomized subjects who completed Day −1 (pre-treatment) MTT assessments and completed all pharmacodynamic assessments for at least one of the three scheduled post-treatment MTT assessments. One subject from the originally randomized cohort of n=45 that completed pre-treatment MTT did not complete any post-treatment MTT assessments and was excluded from the evaluable cohort. Thus, there were 44 subjects in the evaluable population: 12 subjects in the ITCA 650 10 mcg/day group, 11 subjects in the ITCA 650 20 mcg/day group, 10 subjects in the ITCA 650 40 mcg/day group, and 11 subjects in the ITCA 650 80 mcg/day group. Of all scheduled MTT assessments, 43/44 (98%) were completed on Day 5, 37/44 (84%) on Day 15, and 42/44 (95%) on Day 29.

Data from SAS dataset “LB” containing all lab values were downloaded into an Excel file (2013 v15 Office 365 module) for sorting of plasma glucose, insulin and glucagon values by treatment group, subject, visit number, and time within the meal tolerance assessment (there being 7 values, including 1 pre-meal and 6 post-meal, for each analyte). Assembled Excel tables were imported into GraphPad Prism (v7.02.185, San Diego, CA) for graphical analysis.

Values missing from a time series, where there was a preceding and following value, were imputed by linear interpolation. Where an initial value in a time series was missing, it was imputed as the median of the values present at that time point. Since initial values were typically low, the bias from this treatment is likely negligible. The number of values imputed by this method was 11 (of a final matrix of 3611 values; 0.3%).

### Example 1. ITCA-650 and Gastric Emptying Rate

Changes in plasma glucose result from differences in rate of appearance (Ra) and rate of disappearance (Rd; disposal). Rd is primarily an insulin-driven flux. Ra is comprised of meal-related appearance, as well as glucose from endogenous sources, such as hepatic gluconeogenesis. Because insulin is initially low, and takes time to reach its cellular target in the fat and muscle interstitium, and because it takes time to exert its cellular effect of mobilizing GLUT4 transporters, most of the meal-related changes in the initial 30-60 minutes after a meal relate to rates of appearance. Agents that slow the emptying of the stomach, including amylin agonists, CCK agonists, PYY agonists and GLP-1 agonists, dose-dependently suppress glucose rise following test meals, regardless of the effect of such agents to modify insulin secretion. When glucose is the test meal (OGTT), simultaneously measured gastric emptying correlated highly with changes in plasma glucose at 30 min (Horowitz, M., M. A. Edelbroek, J. M. Wishart and J. W. Straathof (1993). “Relationship between oral glucose tolerance and gastric emptying in normal healthy subjects.” Diabetologia 36(9): 857-862). Changes in plasma glucose from pre-meal to 30 minutes post-meal (ΔGlucose₃₀) were explored as evidence of an effect of ITCA-650 on gastric emptying.

### Methods

Changes (ΔGlucose₃₀) were related to those observed before treatment, and the difference (ΔΔGlucose₃₀) explored as a function of duration of treatment and exenatide infusion rate. Dose responses were fitted to a 3-parameter sigmoid (GraphPad Prism v7; San Diego CA), and the fits constrained so that the dose-responses from each of the 3 durations of treatment (5, 15 and 29 days) shared a common ED₅₀.

### Results

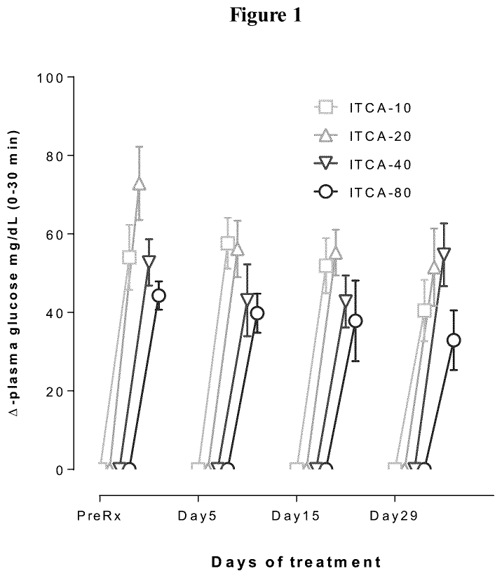

The ΔGlucose₃₀ for each dose group, before and after 5, 15 and 29 days of treatment are shown in FIG. 1 which illustrates 0-30-minute increments in plasma glucose during test meals for 10-, 20-, 40- and 80 μg/day exenatide treatments, measured before and after 5, 15, and 29 days of treatment. Symbols are group means of individual increments±standard error of the mean (SEM).

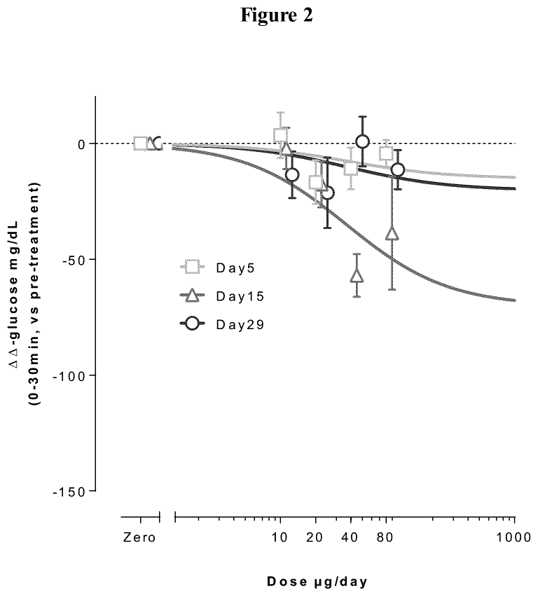

The ΔΔGlucose₃₀, representing the pretreatment-referenced change, is plotted as a function of dose in FIG. 2. A dose-dependency of ΔGlucose₃₀ was suggested after 15 days of treatment (r² 0.22), but this was not apparent either before, at Day 5 (r² 0.02) or after, at Day 29 (r² 0.01).

FIG. 2 illustrates dose-responses for 30-minute changes in glucose concentrations during test meals relative to pre-treatment values. Curves for Days 5, 15 and 29 are 3-parameter sigmoids constrained to share a common ED₅₀. Symbols are group means of individual values±SEM.

### Exemplary Conclusions

Changes in plasma glucose after a test meal, as shown in FIG. 1, were of the order of 40 to 60 mg/dL 30 minutes after the meal. The increments after treatment were similar to the values recorded in the same subjects prior to treatment.

A dose-dependency of changes relative to those observed prior to treatment was suggested after 15 days of treatment, but was not present after either 5 or 29 days of treatment.

The magnitude of suppression of post-meal glucose increments, where present, was small compared to another study in non-diabetic subjects where changes in post-meal glucose were measured following s.c. bolus injections of 5 or 19 μg exenatide (Linnebjerg, H., P. A. Kothare, Z. Skrivanek, A. de la Pena, C. Ernest, M. Atkins and M. E. Trautmann (2004). “Exenatide: postprandial glucose pharmacodynamics at various dosing times relative to a meal in patients with type 2 diabetes.” Diabetologia 47(suppl 1): A280. Abstract 776). The exenatide dose-dependency observed in that study, and in another where glucose was the test meal (OGTT) (Kolterman, O. G., J. B. Buse, M. S. Fineman, E. Gaines, S. Heintz, T. A. Bicsak, K. Taylor, D. Kim, M. Aisporna, Y. Wang and A. D. Baron (2003). “Synthetic exendin-4 (exenatide) significantly reduces postprandial and fasting plasma glucose in subjects with type 2 diabetes.” J Clin Endocrinol Metab 88(7): 3082-3089) was not a consistent feature in the current study.

Without being bound by theory, it thus appears that the effect of bolus injections of exenatide on post-prandial glucose changes may be, at least in part, a consequence of inhibition of gastric emptying. By contrast, gastric emptying does not appear to be inhibited upon chronic infusion of exenatide, as in the present study.

### Example 2. ITCA-650 and Post-Prandial Glucagon Secretion

Exaggeration of glucagon secretion in response to protein-containing meals has been reported in subjects with insulinopenic diabetes, including severe type 2 diabetes (Raskin, P., I. Aydin, T. Yamamoto and R. H. Unger (1978). “Abnormal alpha cell function in human diabetes: the response to oral protein.” Am J Med 64(6): 988-997) and has been implicated in the pathogenesis of disturbed metabolism (Unger, R. H. (1978). “Role of glucagon in the pathogenesis of diabetes: the status of the controversy.” Metabolism 27(11): 1691-1709).

### Methods

Plasma glucagon concentration profiles during meal tolerance tests were plotted as a function of treatment (10-, 20-, 40- and 80-fig exenatide per day) and as a function of duration of treatment (pre-treatment and after 5, 15 and 29 days of treatment). Means and SEM of the data at each of these 16 conditions (4 treatments×4 durations) was derived from data present with no imputation of missing values. Numbers of values present ranged from 7-12.

Data were also analyzed as absolute change from baseline (Δglucagon), and plotted as for glucagon for each of the 16 conditions.

Area under the curve for total glucagon (AUC₀₋₃) and for change in glucagon from 0 min during the MTT (ΔAUC₀₋₃) were derived by trapezoidal interpolation and were each plotted as a function of duration of treatment for each of the treatment groups.

### Results

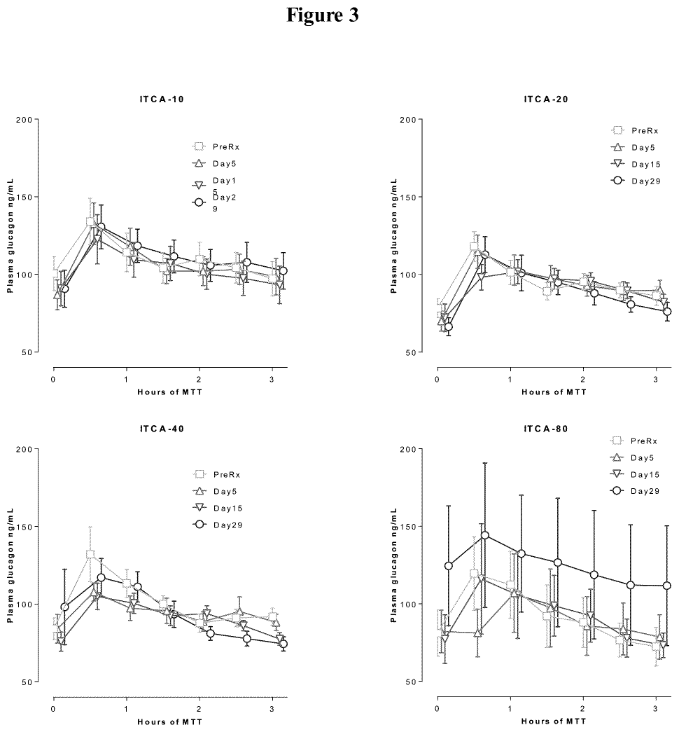

Plasma glucagon profiles during meal tolerance tests are plotted as a function of duration of treatment, for each dose group in separate panels, in FIG. 3. Plasma glucagon profiles were typically maximal 30 min after the test meal, declining gradually thereafter. The profiles were similar between all 16 treatments shown. A high initial baseline and high SEM in the 80-μg/day treatment group at Day 29 was driven by 2 subjects with values 4- to 6-fold higher than values in the other 15 treatment conditions, and may not be reliable.

FIG. 3 illustrates plasma glucagon profiles during meal tolerance tests plotted according to duration of treatment (different symbols and colors) for each of the 4 dose groups (separate panels). Symbols are means±SEM for data present at each condition.

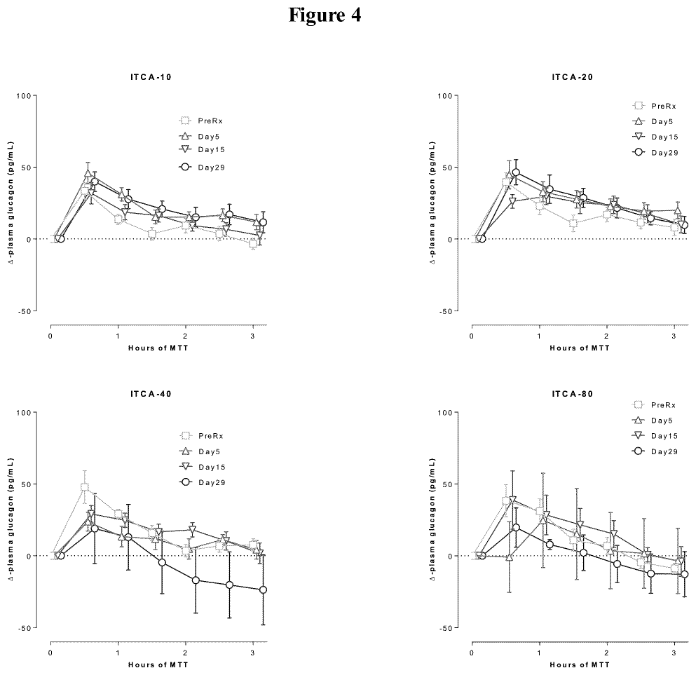

Change in plasma glucagon from pre-meal values is plotted in FIG. 4. Profiles were generally similar for each of the 16 conditions. While changes appeared less for 40- and 80-μg/day treatments at Day 29, there was no indication of a suppression of post-prandial glucagon at Day 15. These measures may be unreliable for the reasons addressed above.

FIG. 4 illustrates changes in plasma glucagon concentration from pre-meal values during a test meal. Symbols, colors and layout have the same meanings as those in FIG. 3.

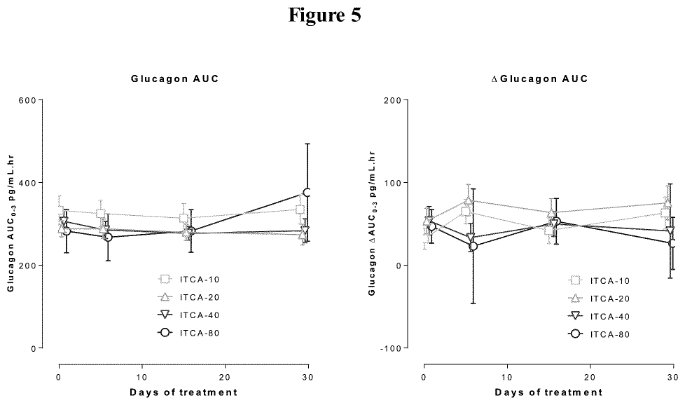

The AUC for absolute glucagon concentrations and for post-meal change in concentration graphed in FIGS. 3 and 4, are plotted in FIG. 5 as a function of duration of treatment for each of the 4 dose groups.

By neither analysis does there appear to be a change from pre-treatment AUC₀₋₃ or ΔAUC₀₋₃ at any duration of treatment.

FIG. 5 illustrates integrated glucagon concentrations (left panel) or glucagon changes (right panel) during Meal Tolerance Test (MTT) as a function of duration of treatment for each dose group.

### Exemplary Conclusions

The data obtained for continuous subcutaneous infusions of exenatide with ITCA-650 do not support suppression of post-prandial glucagon as a significant mechanism underlying its glucose-lowering effect. These observations contrast with those of Kolterman et al. (Kolterman, et al., J Clin Endocrinol Metab 2003) where bolus subcutaneous injections of 1-μg/kg exenatide abrogated the ˜70 pg/mL increase in plasma glucagon 1 hour after a test meal. Since meal-stimulated glucagon secretion may be at least partially moderated by changes in gastric emptying, the absence of effect here may be consistent with an absence of effect of continuously delivered exenatide on gastric emptying, as described above.

### Example 3. ITCA-650 and Glucose-Stimulated Insulin Secretion

The ability of glucagon-like peptide-1 was reported in 1987 (Mojsov, S., G. C. Weir and J. F. Habener (1987). “Insulinotropin: glucagon-like peptide I (7-37) co-encoded in the glucagon gene is a potent stimulator of insulin release in the perfused rat pancreas.” J Clin Invest 79(2): 616-619) to stimulate insulin secretion in a glucose-dependent manner, having no effect at low plasma glucose concentrations. Every GLP-1 agonist reported since then appears to have this property. We therefore sought to determine whether the relationship between resulting plasma insulin concentrations and simultaneously determined plasma glucose concentrations in the present study supported such a mechanism.

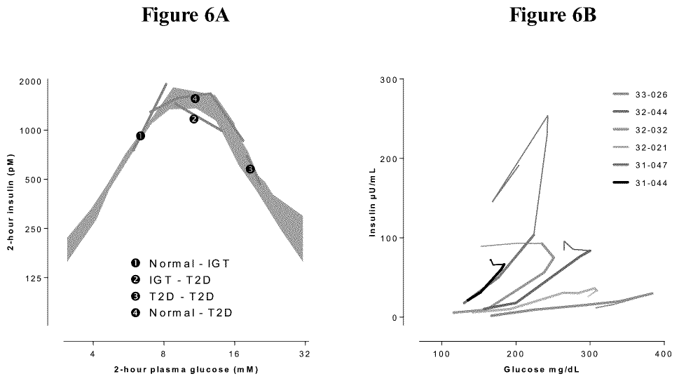

A challenge arises in determining the [insulin]/[glucose] relationship in subjects with type 2 diabetes because the natural history of T2D places subjects in different zones of the [insulin]*[glucose] plane, according to the stage of their disease. Proposed by Reaven and Miller (Reaven, G. M. and R. Miller (1968). “Study of the relationship between glucose and insulin responses to an oral glucose load in man.” Diabetes 17(9): 560-569) based upon cross-sectional data, and affirmed by Saad et al. (Saad, M. F., W. C. Knowler, D. J. Pettitt, R. G. Nelson, D. M. Mott and P. H. Bennett (1989). “Sequential changes in serum insulin concentration during development of non-insulin-dependent diabetes.” Lancet 1(8651): 1356-1359) based upon longitudinal data, the progression begins with amplification of insulin secretion, accompanied by moderate dysglycemia, as insulin resistance becomes established. This is followed in a subset of individuals by florid hyperglycemia, as insulin secretory capacity fails, likely due to islet destruction by amyloid. The result is an inverted U-shaped distribution of [insulin]/[glucose] data pairs, shown for the 2-hour post-OGTT timepoint in FIG. 6A. Individuals tend to follow the trajectories of the yellow arrows as they progress from normal, to IGT, to T2D. FIG. 6B, shows [insulin]/[glucose] diagrams from the MTT in T2D subjects prior to treatment with 80-μg/day exenatide. The progression mapped in FIG. 6A is apparent in the [insulin/[glucose] diagram from the current study in FIG. 6B. The sequence of serial measurements is indicated by the direction of the arrows. For example, subjects 31-047 and 31-044 show a vigorous insulin response with modest increases in glucose following the test meal, consistent with the insulin resistant phase of progression. In contrast, subjects 32-021 and 33-026 show large glycemic excursions and only meager insulin responses, consistent with the secretory failure phase of disease progression. Another feature of the [insulin]/[glucose] trajectories in the current study is hysteresis, wherein the path of descending data pairs is different from that of ascending data pairs. Accommodation of these features is addressed in the analytic methods.

FIG. 6A (left), redrawn from Saad et al., maps the changing [insulin] vs [glucose] relationship during the progression from normal glucose tolerance to T2D. FIG. 6B (right) exemplifies the diverse [insulin] vs [glucose] relationships in the current study.

### Methods

The effect of glucose upon insulin secretion was quantified as the slope of the [insulin] vs [glucose] relationship, as exemplified in FIG. 6B. The slope was estimated by linear regression, the intersection with the X-axis being unique for each subject.

Because of factors such as the time lag for induction of insulin effect, and non-instantaneous clearance of secreted insulin, only data pairs for the ascending part of the hysteresis loop were used in the analysis. These segments are signified by the thick lines in FIG. 6B. Thus, subjects 31-044 and 31-047 yielded similar slopes. Subjects 32-021 and 32-032 had similar slopes but different intersections with the X-axis. Subject 33-026 had the lowest slope.

Such diagrams were analyzed for each subject for each meal tolerance test (pre-treatment and after 5, 15 and 29 days of treatment). Observation suggested that the X-intercept (glucose concentration below which insulin was not secreted) was essentially unchanged by the treatments, so linear regression was constrained to yield a best-fitting fixed X-intercept for all tests in a given subject. Families of up to 4 [insulin] vs [glucose] relationships were fitted to a straight line where the X-intercept was shared, but slopes were able to vary. This was done by fitting the equation [glucose]=m.[insulin]+c (actually the inverse of slopes in FIG. 6B) using least squares interaction in the non-linear module of Prism v7 (San Diego, CA), and retrieving the reciprocal of m as the [insulin] vs [glucose] slope.

Because pre-treatment slopes varied widely between individuals, slopes derived during treatment were expressed as a multiple of the pre-treatment slope. Negative slopes, comprising 4/216 (1.8%) of those derived, were disregarded.

### Results

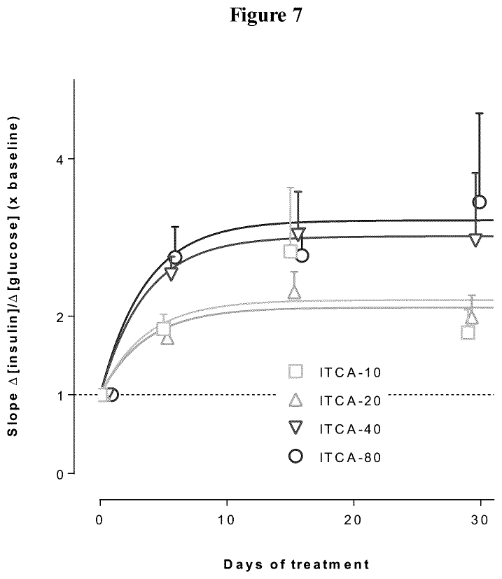

The slope of the [insulin]/[glucose] relationship increased from 1.7-fold with 10 μg/day treatment up to 3.45-fold with 80 μg/day treatment. The slope was near maximal after a week (tau 3.5 days), as shown in FIG. 7.

FIG. 7 illustrates multiples above pre-treatment baseline of best fitting [insulin]×[glucose] slopes. The curves are the best fitting exponential association as a function of duration of treatment.

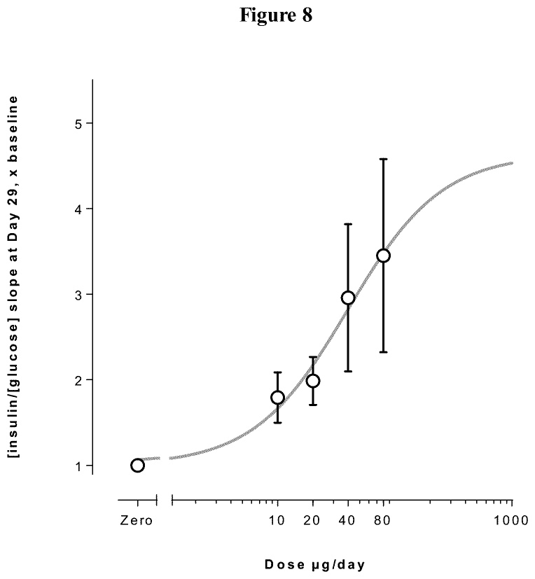

The relative increments in slope after 29 days were analyzed by dose group to obtain the dose response relationship shown in FIG. 8. The sigmoid fit suggests the ED 50 for the slope change is ˜40 μg/day.

FIG. 8 illustrates dose response for the effect of ITCA-650 to increase slope of the [insulin]/[glucose] relationship.

### Exemplary Conclusions

Analysis of treatment-related changes in [insulin] vs [glucose] relationships during meal tolerance tests are indicative of an insulinotropic effect of ITCA-650. Dose response analysis indicates this effect is dose dependent, and that the ED 50 may be near or below indicated doses.

### Example 4. ITCA 650 and the Pharmacokinetics (PK) of Acetaminophen (APAP) and Other Commonly Co-Administered Drugs

### Methods

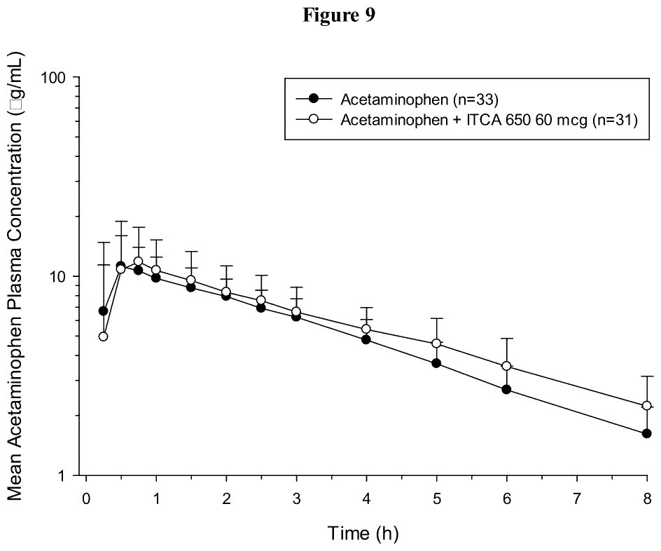

Thirty-three (33) healthy volunteers were enrolled in a sequential, open-label study to assess the effect of ITCA 650 on the PK of APAP 1000 mg, and on the PK and pharmacodynamics (PD) of 4 commonly co-administered drugs: atorvastatin (40 mg), lisinopril (20 mg), digoxin (0.5 mg), and warfarin (25 mg) administered as a cocktail. See FIG. 9. APAP, a marker of gastric emptying, was administered on Day (D)1 followed by the cocktail on D2. ITCA 650 20 mcg/day was placed on D6 and replaced by ITCA 650 60 mcg/day on D20. APAP was administered again on D27 and the cocktail on D28. ITCA 650 60 mcg/day was removed on D32. Serial PK (exenatide; co-administered drugs) and PD (PT-INR) samples were collected.

### Results

There was minimal effect of ITCA 650 on gastric emptying rate as seen in FIG. 9 with the 90% CI of the LS means ratio for AUC between 80-125%. There were no changes in digoxin and warfarin PK or INR. While there were moderate increases in lisinopril and atorvastatin exposures, there were no clinically relevant effects on safety and tolerability of either drug.

### Exemplary Conclusion

There was no substantial effect of ITCA 650 on gastric emptying and no dosage adjustment is deemed necessary when ITCA 650 is co-administered with these commonly used drugs.

### Example 5. ITCA 650 and the PK and Pharmacodynamics (PD) of a Combination Oral Contraceptive (OC)

### Methods

Twenty-eight (28) healthy premenopausal women on a stable regimen of an OC participated in a randomized, double-blind, placebo-controlled, 2-period crossover study. The effect of ITCA 650 on the steady-state PK of ethinyl estradiol (EE) and levonorgestrel (LNG) from Levora® (OC) were evaluated. The study included a 2-week run-in on Levora and 2 treatment periods of 28 days each. In Period 1, ITCA 650 20 mcg/day or ITCA placebo was placed on Day (D) 1 followed by removal and replacement with ITCA 650 60 mcg/day or ITCA placebo on D15. Subjects were crossed over to the alternative treatment and procedures were repeated in Period 2. The OC was administered daily through D28 of each period. Serial samples for PK analysis of exenatide, EE, LNG, and pharmacodynamics (LH, FSH, and progesterone) analysis were collected.

### Results

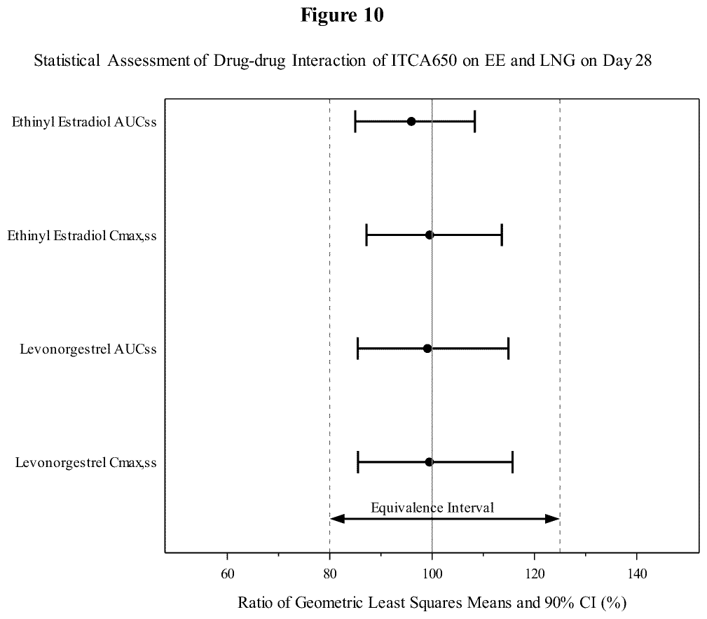

No effect of ITCA 650 60 mcg/day on EE and LNG PK was observed (FIG. 10). The 90% CIs of the geometric LS mean treatment ratios for AUCₛₛ and C_{max,ss} were contained within the equivalence limits of 80% to 125%. Levels of LH, FSH, and progesterone were unaffected by the administration of ITCA 650.

### Exemplary Conclusion

No dose adjustments are required when ITCA 650 is administered with Levora, a combination OC.

### Example 6. Drug Interaction Studies—Potential for Exenatide to Influence the Pharmacokinetics of Other Drugs

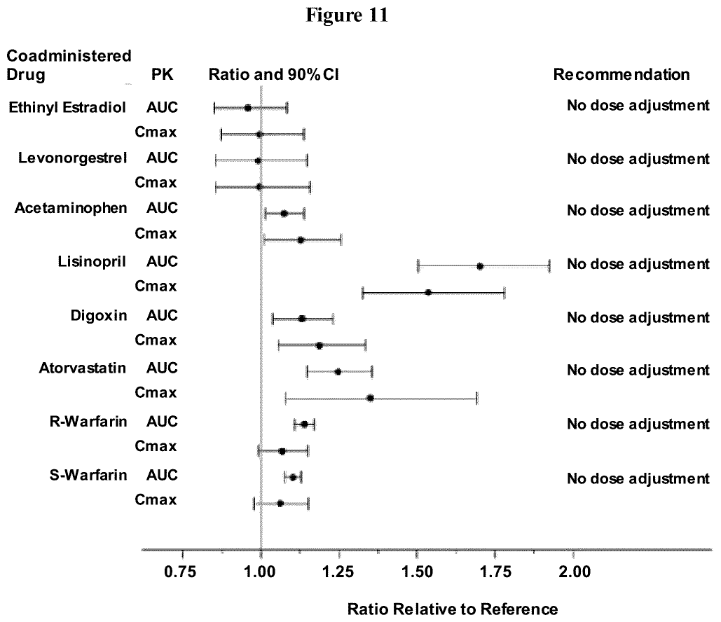

In clinical pharmacology studies ITCA-650 did not affect the pharmacokinetics of the orally administered medications to a clinically relevant degree. FIG. 11 illustrates pharmacokinetic parameters and their 90% confidence intervals (CI), indicating the magnitude of these interactions. No dose adjustment is recommended for any of the evaluated co-administered medications. ITCA-650 had a minimal effect on acetaminophen pharmacokinetics indicating that it has a minimal effect on gastric emptying. ITCA-650 did not significantly alter the pharmacodynamic effects of warfarin as measured by the international normalized ratio (INR).

1. A method comprising
administering to a subject in need thereof, via an implantable delivery device, a continuous subcutaneous dose of an amylin agonist, a PYY agonist, or a glucagon-like peptide-1 (GLP-1) agonist,
wherein:
the subject is orally co-administered a drug after implantation of the implantable delivery device and during continuous subcutaneous dosing of the GLP-1 agonist;
and wherein the drug is administered without dose adjustment for treatment of a disease or disorder other than type-2 diabetes.

2-4. (canceled)

5. The method of claim 1, wherein the drug is selected from the group consisting of acetaminophen, atorvastatin, lisinopril, digoxin, ethinyl estradiol, levonorgestrel, R-warfarin, and S-warfarin.

6. The method of claim 1, wherein the drug is a pain reliever.

7. The method of claim 1, wherein the drug is acetaminophen.

8. The method of claim 1, wherein the drug is an oral contraceptive.

9. The method of claim 1, wherein the drug is one or both of ethinyl estradiol and levonorgestrel.

10. The method of claim 1, wherein the drug is for the treatment of elevated blood levels of cholesterol.

11. The method of claim 1, wherein the drug is a statin.

12. The method of claim 1, wherein the drug is atorvastatin.

13. The method of claim 1, wherein the drug is for the treatment or prevention of hypertension and/or heart disease.

14. The method of claim 1, wherein the drug is digoxin.

15. The method of claim 1, wherein the drug is an angiotensin converting enzyme (ACE) inhibitor.

16. The method of claim 1, wherein the drug is lisinopril.

17. The method of claim 1, wherein the drug is for the treatment or prevention of a heart attack, stroke, hypertension or blood clot.

18. The method of claim 1, wherein the drug is an anticoagulant.

19. The method of claim 1, wherein the drug is R-warfarin and/or S-warfarin.

20-34. (canceled)

35. The method of claim 1, wherein the GLP-1 analog is administered for treatment of a metabolic disorder.

36. The method of claim 1, wherein the GLP-1 analog is administered for treatment of a type 2 diabetes mellitus.

37. The method of claim 1, wherein the GLP-1 analog is administered for treatment of obesity.

38. The method of claim 1, wherein the GLP-1 analog is administered for effecting weight loss in the subject.

39-41. (canceled)


SEQUENCE LISTING (1 sequence)

SEQ ID NO: 1 - protein, 39 aa, Heloderma suspectum
HGEGTFTSDLSKQMEEEAVRLFIEWLKNGGPSSGAPPPS